(19) United States
(12) Patent Application Publication
(10) Pub. No.: US 2015/0273100 A1
(43) Pub. Date: Oct. 1, 2015

(54) FRAGRANCE EMITTING SNOW GLOBE ASSEMBLY

(71) Applicant: Belle-Aire Fragrances, Inc., Mundelein, IL (US)

(72) Inventor: Charles S. David, Mundelein, IL (US)

(21) Appl. No.: 14/663,751

(22) Filed: Mar. 20, 2015

Related U.S. Application Data
(60) Provisional application No. 61/971,014, filed on Mar. 27, 2014.

(51) Int. Cl. A61L 9/12; A61L 9/04; G09F 19/02

(52) U.S. Cl. CPC A61L 9/122; G09F 19/02; A61L 9/04

(57) ABSTRACT

A fragrance emitting snow globe assembly is provided which has the ability to interchange globes with different themes and which has the ability to emit fragrances therefrom that are associated with the theme of the globe being used and displayed. The assembly further being configured to secure and lock the globes in place relative to a base portion of the assembly. The assembly further has the ability to agitate liquid media and particulate material provided within the globe. A method of operating the fragrance emitting snow globe assembly is also provided.

### CROSS-REFERENCE AND INCORPORATION BY REFERENCE

This patent application claims the benefit of domestic priority of U.S. Provisional Patent Application Ser. No. 61/971,014, filed Mar. 27, 2014, and entitled “Snow Globe”, U.S. Provisional Patent Application Ser. No. 61/971,014 is hereby incorporated by reference in its entirety.

### FIELD OF THE INVENTION

The invention relates in general to snow globes. More specifically, the invention relates to snow globes having the ability to interchange globes with different themes and which have the ability to emit fragrances that are associated with (support or enhance, etc.) the theme of the images displayed in the interchangeable globes.

### BACKGROUND OF THE INVENTION

There is an identified need for a new and novel air freshening device for the consumer marketplace. During the 1990's and most of the first decade of this century, candles were the largest growth product for air freshening and ambiance in the home. More recently, reed diffusers and electric heated wall outlet plug-ins have had strong growth. The market, however, is currently saturated with these types of products with numerous players and price pressures from the mass distributors. The introduction of new air freshening products often starts at the top end of distribution with such marketers as Things Remembered®, Yankee Candle Co®, Aromatique® or home party companies such as Party Lites® or Gold Canyon®. A profitable price point of a new product, as well as the opportunity for repeated sales to the same consumer is Important to maintain steady growth and profits. A product that can offer multiple benefits to the customer, promote a brand identity, as well as fit well with a company's core business of the sale of fragrance-related products should be well-accepted. Since the air freshener marketers mentioned promote fragrance as their main product with different “vehicles” to introduce the fragrance to the environment, then a product that can offer them a new concept should prove to be successful. The present invention provides such a concept.

### BRIEF DESCRIPTION OF THE DRAWINGS

The organization and manner of the structure and operation of the invention, together with further objects and advantages thereof, may best he understood by reference to the following description, taken in connection with the accompanying drawings, wherein like reference numerals identify like elements in which:

FIG. 1 is a side view of the snow globe assembly of a preferred embodiment of the invention, which view illustrates the interior portions thereof;

FIG. 2 is a side view of an upper assembly of the snow globe assembly of a preferred embodiment of the invention, where the upper assembly includes a globe and an upper housing, and which view illustrates the interior portions thereof;

FIG. 3 is a side view of a lower assembly of the snow globe assembly of a preferred embodiment of the invention, where the lower assembly includes a lower housing, and which view illustrates the interior portions thereof;

FIG. 4 is a bottom view of the lower assembly of the snow globe assembly;

FIG. 5 is a top view of a drawer-type member used in connection with the snow globe assembly;

FIG. 6 is a top view of a fragrance cartridge used in connection with the snow globe assembly;

FIG. 7 is a side view of a stop member used in connection with the lower assembly of the snow globe assembly;

FIG. 8 is a side view of a first alternative stop member used in connection with the lower assembly of the snow globe assembly; and

FIG. 9 is a side view of a second alternative stop member used in connection with the lower assembly of the snow globe assembly.

### SUMMARY OF THE INVENTION

In a preferred embodiment, the invention provides an assembly comprising a decorative device, a. housing assembly, and a fan assembly. The housing assembly is operatively associated with the decorative device. The housing assembly extends downwardly from the decorative device to a base thereof. The housing assembly defines at least one wall which generally extends from the base to the decorative device. The at least one wall defines first and second aperture sets. The first aperture set is positioned below the second aperture set Each of the first and second aperture sets defines at least one aperture. The housing assembly is further configured to receive and hold a fragrance product therein at a position below the first aperture set. The fan assembly is operatively associated with the housing assembly. The fan assembly includes a rotatable propeller device which is positioned between the first and second aperture sets. The fan assembly is configured to draw air into the housing assembly through the first aperture set, to draw fragrance from the fragrance product upwardly, to mix the drawn-in air and the fragrance together, and to propel the mixed air and fragrance out of the housing assembly through the second aperture set.

In another preferred embodiment, the invention provides an assembly comprising and upper assembly and a lower assembly. The upper assembly comprises an upper housing and a globe. The upper housing and the globe are operatively associated with one another. The upper housing has a stop member associated with it. The globe has a liquid media and a quantity of a particulate material provided therein, as well as one or more objects mounted therein. The objects and/or the quantity of particulate material define a theme of the globe. The lower assembly comprises a lower housing having a stop member associated therewith. The stop member of the lower housing has at least one guide slot associated therewith. The upper and lower housings are configured to be secured to, and removable from, one another. The upper and lower housings are secured to one another when the stop member of the upper housing is positioned in a desired position relative to the stop member of the lower housing. The at least one guide slot of the lower housing is configured to receive and lock the stop member of the upper housing therein.

In another preferred embodiment, the invention provides a fragrance emitting snow globe assembly comprising a base assembly, a first themed set and a second themed set. The first themed set is configured to be operatively associated with the base assembly during a first predetermined time period. The first themed set comprises a first snow globe and a first fragrance product. The second themed set is configured to be operatively associated with the base assembly during a second predetermined time period. The second themed set comprises a second snow globe and a second fragrance product. A theme of the first themed set is different from a theme of the second themed set. The first predetermined time period is different than the second predetermined time period.

In another preferred embodiment, the invention comprises a method of operating a fragrance emitting snow globe assembly. The method comprises the steps of providing a base assembly; providing a first themed set having a first snow globe and a first fragrance product, the first snow globe containing a liquid media, a particulate material, and an agitating device therein; providing a second themed set having a second snow globe and a second fragrance product, the second snow globe containing a liquid media, a particulate material, and an agitating device therein; operatively associating the first themed set with the base assembly during a first predetermined time period by connecting the first snow globe to the base assembly and by positioning the first fragrance product within the base assembly; during the first predetermined time period, causing the base assembly to move the agitating device within the first snow globe in order to agitate the liquid media and particulate material therein; during the first predetermined time period, causing the base assembly to draw air therein, to mix the drawn-in air with fragrance from the first fragrance product, and to emit the mixed air and fragrance from the base assembly; disassociating the first themed set from the base assembly by disconnecting the first snow globe from the base assembly and by removing the first fragrance product from the base assembly; operatively associating the second themed set with the base assembly during a second predetermined time period by connecting the second snow globe to the base assembly and by positioning the second fragrance product within the base assembly; during the second predetermined time period, causing the base assembly to move the agitating device within the second snow globe in order to agitate the liquid media and particulate material therein; and during the second predetermined time period, causing the base assembly to draw air therein, to mix the drawn-in air with fragrance from the second fragrance product, and to emit the mixed air and fragrance from the base assembly.

### DETAILED DESCRIPTION OF THE ILLUSTRATED EMBODIMENTS

While the invention may be susceptible to embodiment in different forms, there is shown in the drawings, and herein will be described in detail, specific preferred embodiments with the understanding that the present disclosure is to be considered an exemplification of the principles of the invention, and is not intended to limit the invention to that as illustrated and described herein.

Snow globes have been a proven favorite product throughout the past 40-50 years. The gifting of upscale and/or personalized globes as well as the sentimental aspect of them and the images promoted within the globes assure that snow globes are held as endeared products to many consumers. Introducing the ability of a snow globe that promotes fragrances that are complimentary to the theme of the image within the globe will lend both an olfactive as well as a visual scene to create an ambiance equal to a candlelit room. Examples of the combinations that could be promoted might be: (a) a Christmas themed globe coupled with fragrances of apple spice, peppermint or vanilla; (b) an ocean front cottage themed globe coupled with fragrances of sea air or ocean breeze; and (c) an autumn/fall/harvest themed globe coupled with a pumpkin spice or other complementary fragrances. The present invention provides such a snow globe.

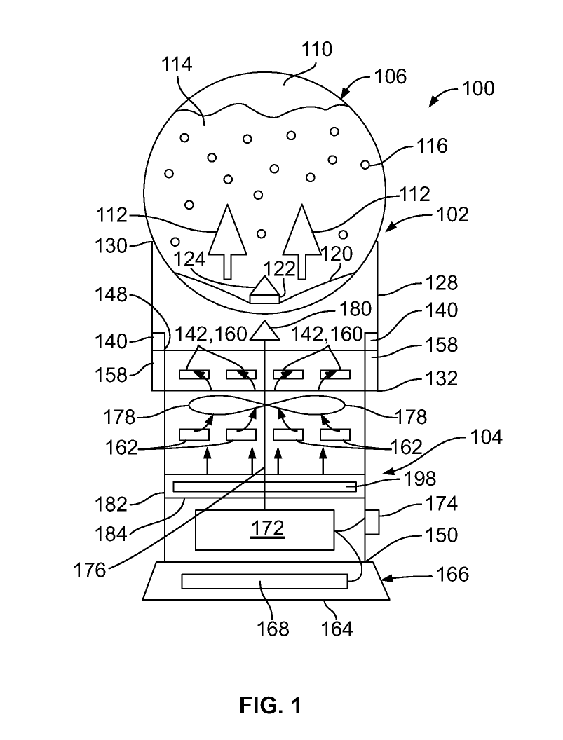

A snow globe assembly 100 of the present invention is best illustrated in FIG. 1. The snow globe assembly 100 has an upper assembly 102 and a lower assembly 104 which are configured to connect with one another in order to allow for the snow globe assembly 100 to function in its desired manner. With regard to a commercial aspect of the snow globe assembly 100, it is intended that the upper and lower assemblies 102, 104 of the snow globe assembly 100 would be purchased together initially and, thereafter, the purchaser of the snow globe assembly 100 could purchase different upper assemblies 102 for use and connection with the originally purchased lower assembly 104.

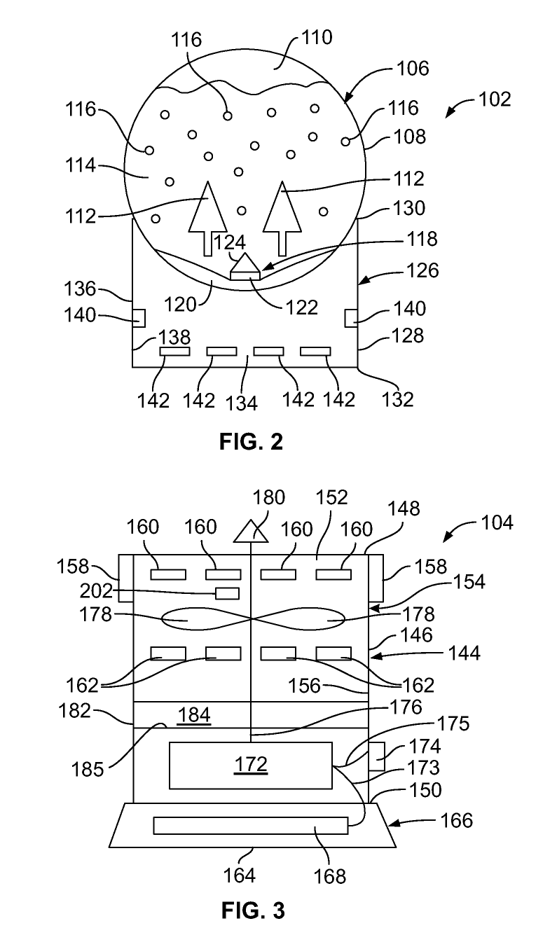

The upper assembly 102 is best illustrated in FIG. 2. The upper assembly 102 includes a globe 106. The globe 106 preferably has at least one outer wall 108 that defines an interior space 110 of the globe 106. The outer wall 108 is preferably spherical or the like in configuration such that the outer wall 108 comprises a single continuous wall, but the outer wall 108 could take the form of other shapes as well and, therefore, the outer wall 108 may not be continuous, but rather may be comprised of multiple walls. The outer wall 108 is preferably formed of, or substantially formed of, a transparent material, preferably glass or plastic, in order to allow for viewing through the outer wall 108 and into the interior space 110 of the globe 106.

One or more objects 112 are provided within the interior space 110 of the globe 106. The object(s) 112 may be provided in any desired shape or configuration and may depict any number of desired images, such as images of people, places or things. The object(s) 112 are preferably mounted within the interior space 110 of the globe 106 in a known manner, such that if the globe 106 is moved (for instance by shaking it back and forth or by turning it upside down), the object(s) 112 will remain in place within the interior space 110 of the globe 106.

The interior space 110 of the globe 106 is also preferably substantially filled with a liquid media 114 and a quantity of a particulate material 116. The object(s) 112 are preferably formed of a material which will not erode or corrode from being subjected to, and positioned within, the liquid media 114 and the particulate material 116. The particulate material 116 may be temporarily suspended in the liquid media 114 upon disturbance of movement of the liquid media 114. The particulate material 116 is often shaped and/or colored to resemble snow or snowflakes, but can also be shaped/colored to resemble any desired image, such as leaves, confetti, balls, shamrocks, etc., as is appropriate. The shape/color of the particulate material 116 is often complimentary to the images displayed on the object(s) 112 in order to provide an overall theme for the globe 106. The themes of the object(s) 112 within the globe 106 may be of a large variety, but are envisioned to primarily be holiday themes, seasonal themes, geographic themes, and sport themes. Like the globe 106, the liquid media 114 is preferably of a type that is transparent in order to allow for viewing through the outer wall 108 and into the interior space 110 of the globe 106, namely to allow for the viewing of the object(s) 112 and the particulate material 116. When the liquid media 114 is agitated, the particulate material 116 relatively slowly descends through the liquid media 114 in order to simulate, for example, snow or leaves falling onto and around the object(s) 112 provided in the interior space 110 of the globe 106.

The interior space 110 of the globe 106 further houses a spinner assembly 118. The spinner assembly 118 is provided at a base 120 of the globe 106 and the interior of the globe 106 is configured to slope or curve downwardly to the base 120. The spinner assembly 118 comprises a magnetic, metal plate 122 and a spinner 124 extending upwardly therefrom. The spinner 124 is preferably formed of a plastic material. The spinner assembly 118 is designed to function by having the spinner 124 rotate upon excitement of the magnetic, metal plate 122. Rotation of the spinner 124 will cause agitation of the liquid media 114 and the particulate material 116 within the interior space 110 of the globe 106, thereby causing the particulate material 116 to cause the desired simulation thereof.

The upper assembly 102 includes an upper housing 126. The upper housing 126 is preferably formed of at least one vertical wall 128. The vertical wall 128 is preferably cylindrical or the like in configuration such that the vertical wall 128 comprises a single continuous wall, but the vertical wall 128 could take the form of other shapes as well and, therefore, the vertical wall 128 may not be continuous, but rather may be comprised of multiple walls. The configuration of the vertical wall 128 is generally dependent on the shape of the outer wall 108 of the globe 106. The vertical wall 128 is preferably formed of a non-transparent material.

The vertical wall 128 of the upper housing 126 is configured to be secured to the globe 106 in any appropriate manner. In a preferred embodiment, an upper edge portion 130 of the vertical wall 128 is secured to the globe 106 by the use of adhesives or the like. The upper edge portion 130 of the vertical wall 128 is preferably secured to the globe 106 above the base 120 of the globe 106 such that at least the base 120 of the globe 106 is positioned within the upper housing 126. In this manner, the spinner assembly 118 is preferably positioned below the upper edge portion 130 of the vertical wall 128, such that the spinner assembly 118 will not be readily visible through the outer wall 108 of the globe 106, and will not detract from the viewing experience afforded by the object(s) 112 and the particulate material 116.

The vertical wall 128 preferably extends downwardly from the upper edge portion 130 to a lower edge portion 132. The lower edge portion 132 defines an opening 134 into the upper housing 126. The vertical wall 128 further defines an outer wall surface 136 and an inner wall surface 138. The outer wall surface 136 may have a design provided thereon if desired. The inner wall surface 138 has at least one stop member 140 associated therewith which is positioned between the upper and lower edge portions 130, 132 of the vertical wall 128. The vertical wall 128 further has at least one aperture 142, and preferably multiple apertures 142, provided therethrough proximate to the lower edge portion 132.

The lower assembly 104 is best illustrated in FIGS. 3-6. As best illustrated in FIG. 3, the lower assembly 104 includes a lower housing 144. The upper housing 126 and the lower assembly 104 may be collectively referred to as the base assembly. The lower housing 144 is preferably formed of at least one vertical wall 146. The vertical wall 146 is preferably cylindrical or the like in configuration such that the vertical wall 146 comprises a single continuous wall, but the vertical wall 146 could take the form of other shapes as well and, therefore, the vertical wall 146 may not be continuous, but rather may be comprised of multiple walls. Like the configuration of the vertical wall 128, the configuration of the vertical wall 146 is generally dependent on the shape of the outer wall 108 of the globe 106. The vertical wall 146 is preferably formed of a non-transparent material.

The vertical wall 146 preferably extends downwardly from an upper edge portion 148 to a lower edge portion 150. The upper edge portion 148 defines an opening 152 into the lower housing 144. The vertical wall 146 further defines an outer wall surface 154 and an inner wall surface 156. Like the outer wall surface 136, the outer wall surface 154 may have a design provided thereon if desired. The outer wall surface 154 has at least one stop member 158 associated therewith which is positioned at or proximate to the upper edge portion 148 of the vertical wall 146. The vertical wall 146 further has at least one aperture 160, and preferably multiple apertures 160, provided therethrough proximate to the upper edge portion 148. The vertical wall 146 further has at least one aperture 162, and preferably multiple apertures 162, provided therethrough between the at least one aperture 160 and the lower edge portion 150.

The lower edge portion 150 of the vertical wall 146 may form a bottom 164 of the lower assembly 104, but more preferably the lower edge portion 150 of the vertical wall 146 rests upon, and is secured to, or integrally formed with, a base 166 of the lower assembly 104. If provided, the base 166 is preferably wider than the lower housing 144, in order to provide stability to the snow globe assembly 100. The bottom 164 of the lower assembly 104 is also provided by the base 166. When the base 166 is provided, an interior of the base 166 is preferably open and in communication with an interior of the lower housing 144.

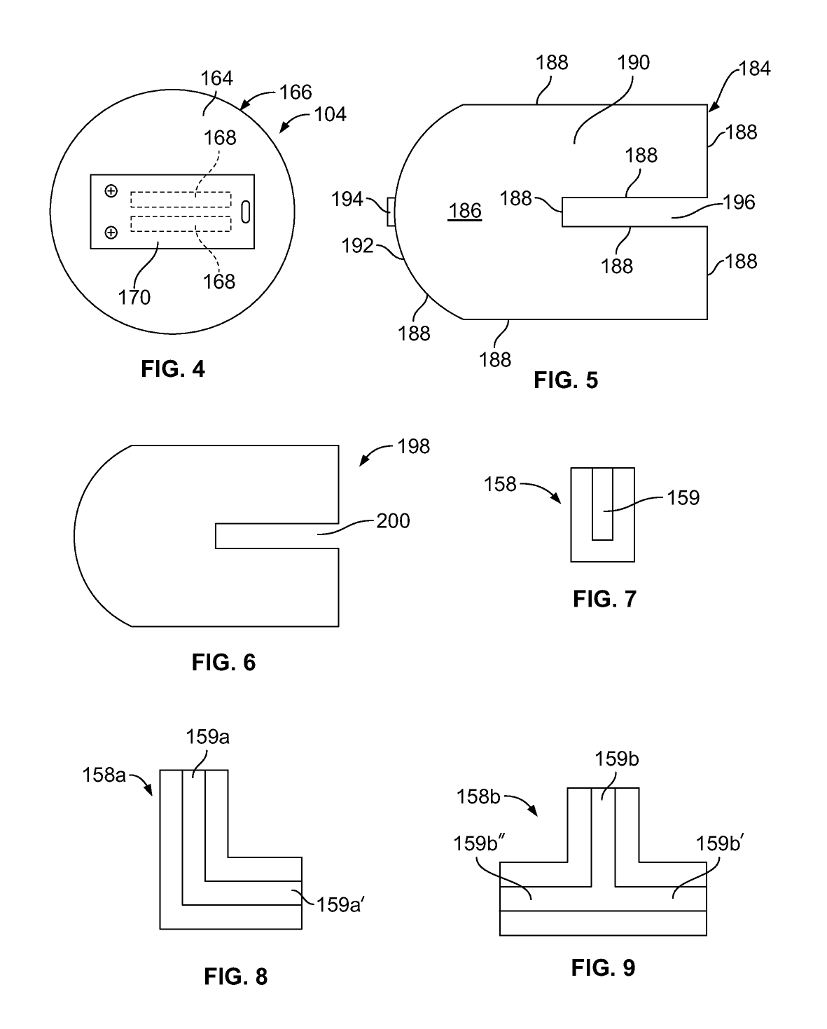

The lower assembly 104 is configured in a known manner to house one or more batteries 168. The batteries 168 are preferably housed proximate to the bottom 164. The batteries 168 may be of any appropriate type, but are preferably of the AA or AAA configuration. As batteries 168 die over time, the lower assembly 104 is preferably configured to provide a battery replacement panel 170, as best illustrated in FIG. 4. The battery replacement panel 170 can be configured to be removed from, and then reattached to, the lower assembly 104 in a known manner in order to allow for the batteries 168 to be replaced. The battery replacement panel 170 is preferably provided in the bottom 164 of the lower assembly 104, regardless of whether the bottom 164 is defined by the lower housing 144 or the base 166.

As best illustrated in FIG. 3, a motor 172 is housed within the lower assembly 104, preferably in a position above the batteries 168. The motor 172 is operatively associated with the batteries 168, preferably by wiring 173, so that the batteries 168 supply power to the motor 172. A switch 174 is preferably provided on an exterior of the lower assembly 104 and is operatively associated with the batteries 168, preferably by wiring 175, so that the switch 174 can be positioned to either allow for the batteries 168 to supply power to the motor 172, or to stop the batteries 168 from supplying power to the motor 172.

The motor 172 has a motor shaft 176 connected thereto which extends upwardly within the lower assembly 104 to approximately the upper edge portion 148 of the lower housing 146. The motor shaft 176 is rotated upon power being supplied to the motor 172. The motor shaft 176 preferably has at least one propeller or blade (or any other suitable fan device) 178 extending therefrom. The propeller 178 is preferably positioned between the apertures 160 and the apertures 162 of the vertical wall 146 of the lower housing 144.

A magnet 180 is secured to an upper end of the motor shaft 176, and the magnet 180 may be positioned proximate to the upper edge portion 148 of the vertical wall 146 of the lower housing 144. The magnet 180 will thus rotate upon rotation of the motor shaft 176.

The vertical wall 146 of the lower housing 144 has an opening 182 provided therethrough in order to allow for the lower housing 144 to receive and house a drawer-type member 184 to be received in, and removed from, the lower housing 144. The inner wall surface 156 of the vertical wall 146 of the lower housing 144 is also configured to provide for a shelf 185 or other support members that would allow for the drawer-type member 184 to be received in, removed from, and held in position within, the lower housing 144. The drawer-type member 184 is preferably held is position with the lower housing 144 between the apertures 162 and the motor 172.

As best illustrated in FIG. 5, the drawer-type member 184 preferably has a floor 186 and at least one wall 188 which extends upwardly from a perimeter of the floor 186, thereby providing a drawer-like structure which defines an opening 190 configured to receive and secure an item therein. The at least one wall 188 preferably has at least a portion thereof which acts as an outer face 192 of the drawer-type member 184 and which is configured to match the shape/design of the outer wall surface 154 of the vertical wall 146 of the lower housing 144, in order to provide a clean look to the outward appearance of the snow globe assembly 100. The outer face 192 of the drawer-type member 184 may be configured to have some type of pull-type handle structure 194 associated with it in order to aid in the insertion/removal of the drawer-type member 184.

It is recognized that the drawer-type member 184 may be prevented from full and proper insertion into the lower housing in view of the configuration and positioning of the motor shaft 176. In view of this, if required, the drawer-type member 184 may be provided with a slot or notch 196 through the floor 186, with the walls 188 being configured to extend upwardly from the floor 186 accordingly.

The snow globe assembly 100 further includes a fragrance cartridge 198, which is best illustrated in FIG. 6. The fragrance cartridge 198 is designed to be positioned and held within the opening 190 of the drawer-type member 184. As such, if the drawer-type member 184 is provided with the slot or notch 196, the fragrance cartridge 198 would likewise be configured to have a similar slot or notch 200 provided therethrough. The fragrance cartridge 198 is preferably configured as a plastic tray that contains a gelled fragrance or solid material impregnated with fragrance to eliminate any spillage that might occur if a liquid fragrance were to be used. The gelled fragrance or solid material can provide any desired scent, and the fragrance cartridges 198 can be easily replaced with different cartridges 198 having either the same scent or a different scent.

Operation of the snow globe assembly 100 will now be discussed. In operation, a user would select a desired upper assembly 102 which has the desired object(s) 112 and particulate material 116 housed within the globe 106. For instance, if it is the Christmas season, a user may select an upper assembly 102 having a Christmas theme, which may include objects 112 in the form of Christmas trees, Santa Claus and reindeer, and the particulate material 116 providing the appearance of snowflakes.

When the desired upper assembly 102 is selected, the user would position the desired upper assembly 102 over the lower assembly 104 and then move one or both of the upper and lower assemblies 102, 104 toward one another, such that the upper assembly 102 would receive the lower assembly 104 within the opening 134 of the upper housing 126. The lower assembly 104 would be received within the opening 134 of the upper housing 126 until the stop members 140, 156 abut against one another. It should be noted that, in an effort to aid the user in this process, the upper and lower housings 126, 144 and/or the stop members 140, 158 could be outfitted with one or more complementary guides and guide slots. For example, as shown in FIG. 7, the stop member 158 could have a vertical guide slot 159 provided therein, into which the stop member 140 would be positioned.

When the upper housing 126 and the lower housing 144 are properly positioned, the apertures 142 of the vertical wall 128 of the upper housing 126 will be in alignment with the apertures 160 of the vertical wall 146 of the lower housing 144. Furthermore, the magnetic, metal plate 122 part of the spinner assembly 118 will locate centered at the base 120 of the globe 106 due to its weight and the slope of the base 120. This will always position the spinner assembly 118 in close proximity to the magnet 180 attached to the motor shaft 176. This is illustrated in FIG. 1

Once the upper and lower assemblies 102, 104 are secured to one another, the user can select a fragrance cartridge 198. If desired, the user can select a fragrance cartridge 198 that has a scent that is complementary to the theme of the selected globe 106, for instance spiced apples, Christmas trees, or candy canes. With the appropriate fragrance cartridge 198 selected, the user would insert the fragrance cartridge 198 into the drawer-type member 184.

The drawer-type member 184 would then be inserted into opening 182 of the vertical wall 146 of the lower housing 144. The drawer-type member 184 would be retained in position within the lower housing 144 and the outer face 192 would close off the opening 182 of the vertical wall 146, providing a clean outward appearance for the snow globe assembly 100.

The user would then operate the switch 174 to its “on” position, such that the batteries 168 would provide power to the motor 172. When the motor 172 is powered, the motor shaft 176 will rotate, thereby rotating both the propellers 178 and the magnet 180.

When the propellers 178 are rotated, fragrance from the fragrance cartridge 198 is drawn upwardly toward the propellers 178, as illustrated by arrows in FIG. 1. Furthermore, air is drawn into the lower housing 144 through the apertures 162 of the vertical wall 146, and this air is also drawn upwardly toward the propellers 178, as illustrated by arrows in FIG. 1. The drawn in air and fragrance are then mixed together within the lower housing 144 and are propelled out of the lower housing 144 by the propellers 178 through the aligned apertures 142/160 of the upper and lower housings 126, 144, as illustrated by arrows in FIG. 1. The apertures 162 and 142/160 thus act as inlet and outlet vents, respectively, for the snow globe assembly 100. The vented mixture of air and fragrance will then provide the desired scent to the area in which the snow globe assembly 100 is provided.

When the magnet 180 is rotated, it will excite the magnetic, metal plate 122 of the spinner assembly 118 within the globe 106, as the magnetic, metal plate 122 and the magnet 180 will be positioned in relative close proximity to one another. The excitement of the magnetic, metal plate 122 will cause the spinner 124 of the spinner assembly 118 to spin, thereby causing agitation of the liquid media 114 and the particulate material 116 within the globe 106. Such agitation/excitement of the particulate material 116 causes the particulate material 116 to move about the interior space 110 of the globe 106, thereby giving the desired appearance of the particulate material 116, e.g., falling snow.

When the switch 174 is turned to its “off” position, the batteries 168 stop providing power to the motor 172 and the motor shaft 176 is no longer rotated. Thus, neither the propellers 178 nor the magnet 180 are being rotated. Thus, the fragrance is not being drawn upwardly from the fragrance cartridge 198 and the spinner assembly 118 is not being excited to agitate/excite the liquid media 114 and the particulate material 116.

When the fragrance cartridge 198 is used up, or if the user just desires a new scent, the user can replace the fragrance cartridge 198 with a new fragrance cartridge by pulling the drawer-type member 184 out (preferably by the handle structure 194), removing the old fragrance cartridge 198, adding a new fragrance cartridge 198, and then reinserting the drawer type member 184 back into the lower housing 144.

When the user desires a new theme in the globe 106, for instance if Christmas time is over and Valentine's Day is arriving, the user may opt to replace the upper assembly 102 having a Christmas theme with an upper assembly 102 having a Valentine's Day theme, object(s) 112 such as Cupid, and particulate material 116 in the form of hearts, In order to replace the Christmas themed upper assembly 102 with a Valentine's themed upper assembly 102, the user would first unlock the upper assembly 102 from the lower assembly 104, if applicable, and then separate the upper and lower assemblies 102, 104 from one another (either by holding one stationary and pulling on the other, or by pulling the two apart from one another). The Valentine's themed upper assembly 102 would then be positioned (and locked, if desired) to the lower assembly 104 in the same manner as the Christmas themed upper assembly 102 was, as previously described.

The drawer-type member 184 and the associated fragrance cartridge 198 are preferably configured to act as a separator between the portion of the lower assembly 104 that contains the fragrance vapors drawn upward by the propellers 178, and the electronics (e.g., the motor 172 and the batteries 168) that could become sensitive to aggressive fragrance materials.

It is to be understood that modifications could be made to the snow globe assembly 100, but that these modifications are to be considered within the scope of the invention.

For instance, the snow globe assembly 100 is described and illustrated as being used with a “snow globe”, however, the “snow globe” described and illustrated herein could be any other type of decorative device, as desired, which could also operate and co-exist with the fragrance emitting portion of the assembly. In such a situation, the spinner assembly 118 and the associated magnet 180 would not need to be included in the assembly. For instance, the decorative device could be a rotatable image or three-dimensional character or display.

For instance, the snow globe assembly 100 is described and illustrated as having the lower housing 144 inserted within the upper housing 126. However, the snow globe assembly 100 could be easily modified to have the upper housing 126 be inserted within the lower housing 144.

The configuration of the stop member 158 could also be modified in order to provide one or both of a locking feature and a masking feature. For instance, the stop member 158 could be modified to have an L-shaped configuration, as illustrated in FIG. 8, and with the stop member 158 now being designated as 158a. The stop member 158a has a vertical guide slot 159a and a horizontal guide slot 159a′. With this configuration, the stop member 140 could be inserted first into the vertical guide slot 159a and then, upon rotation of one or both of the upper and lower assemblies 102, 104, the stop member 140 could be inserted second into the horizontal guide slot 159a′, thereby locking the stop member 140 into position. In this configuration, the apertures 142, 160 would preferably only be aligned when the stop member 140 is in its proper position within the horizontal guide slot 159a′.

Furthermore, the stop member 158 could also, for instance, be modified to have an upside-down T-shaped configuration, as illustrated in FIG. 9, and with the stop member 158 now being designated as 158b. The stop member 158b has a vertical guide slot 159b and first and second horizontal guide slot 159b′ and 159b″. With this configuration, the stop member 140 could be inserted first into the vertical guide slot 159b and then, upon rotation in a first direction of one or both of the upper and lower assemblies 102, 104, the stop member 140 could be inserted second into the first horizontal guide slot 159b′, thereby locking the stop member 140 into position. In this configuration, the apertures 142, 160 would preferably only be aligned when the stop member 140 is in its proper position within the horizontal guide slot 159b′. Also in this configuration, the stop member 140 could be inserted first into the vertical guide slot 159b and then, upon rotation in a second direction of one or both of the upper and lower assemblies 102, 104, the stop member 140 could be inserted second into the second horizontal guide slot 159b″, thereby locking the stop member 140 into position. In this configuration, the apertures 142, 160 would preferably be misaligned when the stop member 140 is in its proper position within the horizontal guide slot 159b″.

The switch 174 may also be configured to have further settings than just “on” and “off”. For instance, the switch 174 could be configured to have a position which activates an LED 202 within the snow globe assembly 100 which is used to illuminate the interior space 110 of the globe 106, such that the snow globe assembly 100 could act as a nightlight. Another position of the switch 174 could be provided where the LED is activated, and where the motor 172 is powered, thereby allowing functions of the snow globe assembly 100 to operate simultaneously. Alternatively, the snow globe assembly 100 could have multiple switch positions which control different functional aspects of the snow globe assembly 100. These functional aspects could also be configured by the switch 174 and the motor 172 to having timing elements associated therewith, such that the switch 174 will allow the motor 172 to cycle on/off for predetermined periods of time.

Another modification that could be made is that the snow globe assembly 100 could be configured to be electrically powered, rather than powered by the batteries 168. The snow globe assembly 100 would then obviously need to be configured to have an electrical plug extending therefrom which is to be inserted into a wall socket.

Another modification that could be made is that the fragrance cartridge 198 could be provided in a form other than a gel, as described, such as a solid. The fragrance cartridge 198 is preferably not formed as a liquid for the reasons discussed hereinabove.

While preferred embodiments of the present invention are shown and described, it is envisioned that those skilled in the art may devise various modifications of the present invention without departing from the spirit and scope of the invention.

The invention is claimed as follows:

1. An assembly comprising:
a decorative device; and
a housing assembly which is operatively associated with the decorative device and which extends downwardly from the decorative device to a base thereof, the housing assembly defining at least one wall which generally extends from the base to the decorative device, the at least one wall defining first and second aperture sets, the first aperture set being positioned below the second aperture set, each of the first and second aperture sets defining at least one aperture, the housing assembly further being configured to receive and hold a fragrance product therein at a position below the first aperture set; and
a fan assembly operatively associated with the housing assembly, the fan assembly including a rotatable propeller device which is positioned between the first and second aperture sets, the fan assembly being configured to draw air into the housing assembly through the first aperture set, to draw fragrance from the fragrance product upwardly, to mix the drawn-in air and the fragrance together, and to propel the mixed air and fragrance out of the housing assembly through the second aperture set.

2. The assembly as defined in claim 1, wherein the decorative device is a globe having a liquid media and a quantity of a particulate material provided therein, the globe further having one or more objects mounted therein, the objects and/or the quantity of particulate material defining a theme of the globe.

3. The assembly as defined in claim 2, wherein the globe further has a magnetic spinner assembly positioned in a base of the globe, and wherein the housing assembly has an agitating assembly which includes a motor, a motor shaft, and a magnet secured to the motor shaft, the magnet and the magnetic spinner assembly being positioned proximate to one another such that rotation of the magnet on the motor shaft causes rotation of the magnetic spinner assembly.

4. The assembly as defined in claim 3, wherein the motor and motor shaft of the agitating assembly also act as part of the fan assembly, with the propeller device being secured to and rotatable by the motor shaft.

5. The assembly as defined in claim 3, wherein the motor is powered by one or more batteries, the one or more batteries being positioned in the housing assembly.

6. The assembly as defined in claim 5, wherein the motor is operable by a switch associated with the housing assembly.

7. The assembly as defined in claim 3, wherein the housing assembly comprises an upper housing and a lower housing which are configured to be secured to, and removable from, one another, the globe being operatively associated with the upper housing, the fan assembly and the agitating assembly being operatively associated with the lower housing.

8. The assembly as defined in claim 7, wherein the globe and upper housing are configured to be removed from the lower housing and replaced with a different globe and upper housing, where the different globe and upper housing has a different theme than the original globe and upper housing.

9. The assembly as defined in claim 7, wherein the upper housing has an aperture set, and wherein the lower housing has an aperture set, the aperture sets of the upper and lower housings being in alignment with one another to form the second aperture set when the upper and lower housings are secured to one another.

10. The assembly as defined in claim 9, wherein the upper and lower housings each have stop members associated therewith, the upper and lower housings being secured to one another when the stop member of the upper housing is positioned in a desired position relative to the stop member of the lower housing.

11. The assembly as defined in claim 10, wherein the stop member of the lower housing has at least one guide slot associated therewith for receiving and locking the stop member of the upper housing therein.

12. The assembly as defined in claim 3, wherein the housing assembly defines an opening below the first aperture set, and wherein a drawer-type member is configured to be removably positioned within the opening of the housing assembly, the drawer-type member configured to have an outer face which, when the drawer-type member is positioned within the housing assembly, is generally co-extensive with the at least one wall of the housing assembly, the drawer-type member configured to hold the fragrance product therein.

13. The assembly as defined in claim 12, wherein the drawer-type member has an opening provided therein, the motor shaft positioned to extend through the opening of the drawer-type member.

14. The assembly as defined in claim 13, wherein the opening of the drawer-type member is a slot.

15. The assembly as defined in claim 12, wherein the fragrance product has a configuration which matches a configuration of the drawer-type member, the fragrance product having an opening therein which is in communication with the opening of the drawer-type member when the fragrance product is held by the drawer-type member.

16. The assembly as defined in claim 1, wherein the housing assembly comprises an upper housing and a lower housing which are configured to be secured to, and removable from, one another, the decorative device being operatively associated with the upper housing, the fan assembly being operatively associated with the lower housing.

17. The assembly as defined in claim 16, wherein the decorative device and upper housing are configured to be removed from the lower housing and replaced with a different decorative device and upper housing.

18. The assembly as defined in claim 1, wherein the housing assembly defines an opening below the first aperture set, and wherein a drawer-type member is configured to be removably positioned within the opening of the housing assembly, the drawer-type member configured to have an outer face which, when the drawer-type member is positioned within the housing assembly, is generally co-extensive with the at least one wall of the housing assembly, the drawer-type member configured to hold the fragrance product therein.

19. An assembly comprising:
an upper assembly comprising an upper housing and a globe, the upper housing and the globe being operatively associated with one another, the upper housing having a stop member associated therewith, the globe having a liquid media and a quantity of a particulate material provided therein, the globe further having one or more objects mounted therein, the objects and/or the quantity of particulate material defining a theme of the globe; and
a lower assembly comprising a lower housing, the lower housing having a stop member associated therewith, the stop member of the lower housing having at least one guide slot associated therewith,
wherein the upper and lower housings are configured to be secured to, and removable from, one another, the upper and lower housings being secured to one another when the stop member of the upper housing is positioned in a desired position relative to the stop member of the lower housing, the at least one guide slot of the lower housing being configured to receive and lock the stop member of the upper housing therein.

20. The assembly as defined in claim 19, wherein the at least one guide slot of the lower housing is L-shaped.

21. The assembly as defined in claim 19, wherein the at least one guide slot of the lower housing is T-shaped.

22. The assembly as defined in claim 19, wherein the upper assembly is configured to be removed from the lower assembly and replaced with a different upper assembly, which has a different globe and upper housing, where the different globe and upper housing has a different theme than the original globe and upper housing.

23. The assembly as defined in claim 19, wherein the upper and lower housings form a housing assembly, the housing assembly extends downwardly from the globe to a base thereof, the housing assembly defining at least one wall which generally extends from the base to the globe, the at least one wall defining first and second aperture sets, the first aperture set being positioned below the second aperture set, each of the first and second aperture sets defining at least one aperture, the housing assembly further being configured to receive and hold a fragrance product therein at a position below the first aperture set.

24. The assembly as defined in claim 23, further comprising a fan assembly operatively associated with the housing assembly, the fan assembly including a rotatable propeller device which is positioned between the first and second aperture sets, the fan assembly being configured to draw air into the housing assembly through the first aperture set, to draw fragrance from the fragrance product upwardly, to mix the drawn-in air and the fragrance together, and to propel the mixed air and fragrance out of the housing assembly through the second aperture set.

25. The assembly as defined in claim 24, wherein the globe further has a magnetic spinner assembly positioned in a base of the globe, and wherein the housing assembly has an agitating assembly which includes a motor, as motor shaft, and a magnet secured to the motor shaft, the magnet and the magnetic spinner assembly being positioned proximate to one another such that rotation of the magnet on the motor shaft causes rotation of the magnetic spinner assembly.

26. The assembly as defined in claim 25, wherein the motor and the motor shaft of the agitating assembly also act as part of the fan assembly, with the propeller device being secured to and rotatable by the motor shaft.

27. The assembly as defined in claim 25, wherein the housing assembly defines an opening below the first aperture set, and wherein as drawer-type member is configured to be removably positioned within the opening of the housing assembly, the drawer-type member configured to have an outer face which, when the drawer-type member is positioned within the housing assembly, is generally co-extensive with the at least one wall of the housing assembly, the drawer-type member configured to hold the fragrance product therein.

28. The assembly as defined in claim 27, wherein the drawer-type member has an opening provided therein, the motor shaft positioned to extend through the opening of the drawer-type member.

29. The assembly as defined in claim 28, wherein the fragrance product has a configuration which matches a configuration of the drawer-type member, the fragrance product having an opening therein which is in communication with the opening of the drawer-type member when the fragrance product is held by the drawer-type member.

30. The assembly as defined in claim 23, wherein the upper housing has an aperture set, and wherein the lower housing has an aperture set, the aperture sets of the upper and lower housings being in alignment with one another to form the second aperture set when the upper and lower housings are secured to one another.

31. A fragrance emitting snow globe assembly comprising:
abase assembly;
a first themed set configured to be operatively associated with the base assembly during a first predetermined time period, the first themed set comprising a first snow globe and a first fragrance product; and
a second themed set configured to be operatively associated with the base assembly during a second predetermined time period, the second themed set comprising a second snow globe and a second fragrance product,
wherein a theme of the first themed set is different from a theme of the second themed set, and wherein the first predetermined time period is different than the second predetermined time period.

32. The fragrance emitting snow globe assembly as defined in claim 31, wherein the base assembly includes a housing which is configured to be secured to either the first snow globe or the second snow globe, and which is configured to house either the first fragrance product or the second fragrance product.

33. The fragrance emitting snow globe assembly as defined in claim 32, wherein the base assembly includes a system for drawing air into the housing, mixing the air with fragrance from the fragrance product, and emitting the mixed air and fragrance from the housing.

34. The fragrance emitting snow globe assembly as defined in claim 32, wherein each of the first and second snow globes contains a liquid media, a particulate material, and an agitating device therein, and wherein the base assembly includes a system which is operatively associated with the agitating device in order to agitate the liquid media and the particulate material within the respective snow globes.

35. A method of operating a fragrance emitting snow globe assembly, the method comprising the steps of:
providing a base assembly;
providing a first themed set having a first snow globe and a first fragrance product, the first snow globe containing a liquid media, a particulate material, and an agitating device therein;
providing a second themed set having a second snow globe and a second fragrance product, the second snow globe containing a liquid media, a particulate material, and an agitating device therein;
operatively associating the first themed set with the base assembly during a first predetermined time period by connecting the first snow globe to the base assembly and by positioning the first fragrance product within the base assembly;
during the first predetermined time period, causing the base assembly to move the agitating device within the first snow globe in order to agitate the liquid media and particulate material therein;
during the first predetermined time period, causing the base assembly to draw air therein, to mix the drawn-in air with fragrance from the first fragrance product, and to emit the mixed air and fragrance from the base assembly;
disassociating the first themed set from the base assembly by disconnecting the first snow globe from the base assembly and by removing the first fragrance product from the base assembly;
operatively associating the second themed set with the base assembly during a second predetermined time period by connecting the second snow globe to the base assembly and by positioning the second fragrance product within the base assembly;
during the second predetermined time period, causing the base assembly to move the agitating device within the second snow globe in order to agitate the liquid media and particulate material therein; and
during the second predetermined time period, causing the base assembly to draw air therein, to mix the drawn-in air with fragrance from the second fragrance product, and to emit the mixed air and fragrance from the base assembly.
